(19) United States
(12) Patent Application Publication
(10) Pub. No.: US 2016/0175375 A1
(43) Pub. Date: Jun. 23, 2016

(54) PREPARATION OF ORALLY ADMINISTRABLE PHARMACEUTICAL COMPOSITION CONTAINING TRADITIONAL CHINESE MEDICINE FOR HEMATOLOGIC DISEASES

(71) Applicant: YUAN-JEN CHANG, Taichung City (TW)

(72) Inventor: YUAN-JEN CHANG, Taichung City (TW)

(21) Appl. No.: 14/573,185

(22) Filed: Dec. 17, 2014

(51) Int. Cl. A61K 36/898; A61K 36/748; A61K 36/19; A61K 36/81; A61K 36/23; A61K 36/71; A61K 36/896; A61K 36/48

(52) U.S. Cl. CPC A61K 36/898; A61K 36/896; A61K 36/748; A61K 36/48; A61K 36/81; A61K 36/23; A61K 36/71; A61K 36/19

(57) ABSTRACT

Preparation of an orally administrable pharmaceutical composition containing traditional Chinese medicine for hematologic diseases is disclosed. The pharmaceutical composition is for immunotherapy and is made from natural, non-toxic medicinal materials through extraction and other preparing processes. The pharmaceutical composition is helpful to prevent, mitigate and cure cancers.

### BACKGROUND OF THE INVENTION

1. Technical Field

The present invention relates to relates to pharmaceutical compositions for treating hematologic diseases, including hematologic cancers, and more particularly to a method for preparing pharmaceutical compositions for immunotherapy from natural medicinal materials. The disclosed method involves processing the natural medicinal materials according to their respective properties, so as to obtain the best effects on preventing, mitigating and curing hematologic diseases.

2. Description of Related Art

Cancers are diseases incurred by abnormal control mechanism for cell division and cell proliferation. Virtually all kinds of cells have the possibility to become tumor cells. However, most human cancers associate with malignant tumors of epithelial cells. This type covers the majority of cancers, and some of them are common cancers, such as breast cancer, prostate cancer and lung cancer. The known malignant epithelial cell tumors can be categorized into malignant squamous epithelial cell tumors (turned from normal epithelial cells) and malignant adenoma.

For controlling cancers, the currently employed therapies are surgical removal, chemotherapy, radiotherapy, immunotherapy and passive immunotherapy, among others. The determination of therapies depends on the tumor positions, malignant levels, evolution and patients' conditions. Some experimental therapies have been also developed for cancers. The aim is to find cancer treatments that can completely eliminate cancer cells while forming no harm to healthy calls. Surgical removal is only effective when there are no spreading or remote metastases. Chemotherapy is not a perfect solution as it is also toxic to the non-targeted cells. Radiotherapy has the same problem. Briefly, cancers represent various tumors in different kinds of cells, and there is not a “single cure” for all of them. Conventionally, the aforementioned therapies are implemented alternatively or simultaneously according to the progress of the cancers to be treated for optimizing the curative effects. However, removal, chemotherapy and radiotherapy are particularly burdens to a patient's weak body. Moreover, once the metastases happen, it will be difficult to control the progress with any of the known therapies. For all these reasons, how to treat cancers is still a big challenge to the human.

Immunity protects our health and it is activated by leukocytes. In other words, leukocytes are soldiers fighting invaders for us. There are three kinds of leukocytes:

(1) Lymphocytes: It's lymphocytes main duty to identify enemies and call immunocytes to attack the abnormal cells.

(2) Macrophages: they eat any bacteria and abnormal cells upon detection.

(3) Granulocyte: granulocytes are also referred to as phagocytic cells, serving to expel bacteria and abnormal cells.

When lymphocytes, macrophages and granulocyte work together, they can have pathogens and unwanted old cells removed from human body. However, tumor cells and virus-infected cells are not foreign invaders, but our own cells. This prevents our immune system from properly identifying the targets. Thus, it is critical to have our immunocytes activated.

Immune system has three main functions:

(1) Guarding from microbial infection: when body cells are invaded, it can expel the infective source and control the infection.

(2) Rejecting cells from other individuals: it produces antibodies after blood transfusion or organ transplantation to reject the foreign cells.

(3) Removing mutated cells and old tissues: when finding trouble cells or unwanted, old cells, it removes them by phagocytosis.

In addition to proper treatments using surgical removal, chemotherapy and radiotherapy, it is important for a patient having a cancer to improve his/her immunity. This may be achieved by ingesting good food, having good lifestyle, and supplementing nutrition that helps to activate lymphocytes, namely B cells, T cells, and NK cells.

There are thousands cancer cells generated in human body. Immune system keeps monitoring through blood circulation, and once finding cancer cells, it attacks. However, cancer cells are extremely tenacious and cunning They will fight back whenever immunologic inadequacy happens. Immunologic inadequacy means that various immunocytes such as T cells, B cells and NK cells are less active. If the inadequate immunity is not recovered in time to call the immunocytes to fight back, cancers will unavoidably happen.

The principle of immunotherapy for treating cancers is to strengthen our immune system so as to clean all the pathogens, old cells, cancer cells, and virus-infected cells. Immunotherapy has proven to provide good curative effects on maintaining health, curing diseases, and providing nourishment during convalescence, and is known as effective to various diseases, from cough and colds to AIDS and cancers. We can say as long as good immunity is maintained well, the body can cure diseases by itself.

In this regard, regularly taking health supplement products is an effective way. There are many immunity-strengthening products in the forms of orally administrable pharmaceutical compositions available on the market. The active ingredients are digested by macrophages in the small intestines, and act on receptors of cell membrane of most human cells, so as to make some actions taking place in cell nucleus stronger, thereby activating immunocytes.

### SUMMARY OF THE INVENTION

The present invention is an application of cancer immunotherapy. By strengthening the activity of autoimmune cells, the autoimmune functionality can identify and clean cancer cells, thereby maintaining health.

The present invention involves selecting natural medicinal materials, immersing the materials with the presence of base liquor or honey, processing the soaks according to the properties of the respective materials, performing extraction, concentration and other processes, so as to produce orally administrable pharmaceutical compositions having preventing, mitigating and curing effects on hematologic diseases.

### BRIEF DESCRIPTION OF THE DRAWINGS

FIG. 1 is a flowchart of the method of the present invention.

FIG. 2 illustrates the mechanism of the present invention.

### DETAILED DESCRIPTION OF THE INVENTION

The invention as well as a preferred mode of use, further objectives and advantages thereof will be best understood by reference to the following detailed description of illustrative embodiments when read in conjunction with the accompanying drawings.

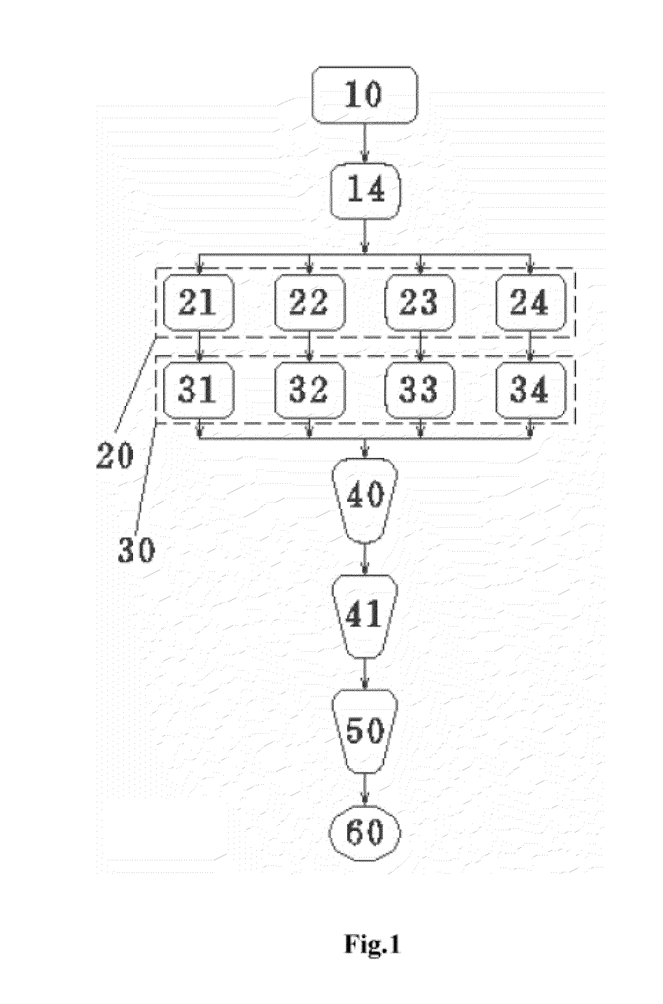

FIG. 1 is a flowchart of the method of the present invention. As shown, selected medicinal materials 10 are first processed by stir-frying 14 and other primary processing processes according to their respective properties, so as to release the active ingredients from the medicinal materials 10. In this step, the medicinal materials that have toxic side effects are reduced in terms of toxicity. The medicinal materials 10 then receive immersion 20 separately under different conditions determined according to their respective properties, so as to further release active substances. As disagglomeration happens in the soak of the immersion 20, each of the medicinal materials 10 has its ingredients balanced by the fermentable substances of the base liquor used in the immersion 20. Then some adjuvants are added into the soaks of the medicinal materials 10 after the immersion 20. The soaks then receive extraction 30, which extracts more active substances from the medicinal materials 10, and chelates the medicinal materials 10 at different temperatures, thereby further enhancing the effects of the extracted substances and obtaining botanical extracts that are nutrimental, gentle, non-toxic and effective. In this manner, each of the medicinal materials 10 is processed to give out its most valuable essentials that are helpful to improve immunity, to enhance health and to cure diseases. The extracts obtained after the extraction 30 are then mixed in a predetermined ratio into a mixture. The mixture receives concentration 40 and forms a concentrate. After filling 41 and sterilization 50, the product 60, or the pharmaceutical composition, is ready to serve.

In the present invention, the selected medicinal materials 10 are processed by stir-frying 14 and other primary processing processes so that the medicinal materials 10 can have their active ingredients released completely, and this helps to reduce amount of the toxic medicinal materials that have toxic side effects. Therein, stir-frying 14 is applied to Semiaquilegia adoxoides(DC.)makino. This process promotes synergism between the medical materials, enhances the curative and detoxifying effects, and modulates the performance of the medical materials. The substance so obtained thus is useful for remedium corrigents, thereby contributing to relieving cough and asthma, and reinforcing the spleen to benefit the qi. The medicinal materials 10 are processed separately during the primary processing stage, and then receive different process of immersion 20. The immersion 20 includes immersion 21 for Baphicacanthus cusia (Nees) Bremek (Natural Indigo), immersion 22 for Mucuna macrocarpa Wallich, immersion 23 for Solanum lyratumThunb (BittersweetHerb) and immersion 24 for Semiaquilegia adoxoides(DC.)makino. A base liquor is used in each said immersion so as to make the medicinal materials 10 release their active substances. The consequent disagglomeration allows the medicinal materials 10 to be sufficiently affected by the fermentable substances in the base liquor and present the most balanced effects.

After the immersion 20, the soaks of the medicinal materials 10 are put into the process of extraction 30. Particularly, among the extraction 30, extraction 31 has Glehnia littoralis Fr:Schmidt ex Miq added, extraction 32 has Hedyotis diffusa added, extraction 33 has Pseudobulb of Appendiculate Cremastra added, and extraction 34 has Parispolyphylla Smith varchinensis(Franch.)Hara added. Thereby, more effective substances can be extracted from the medicinal materials 10, and the medicinal materials 10 are chelated at different temperatures, thereby further enhancing the effects of the extracted substances and obtaining botanical extracts that are nutrimental, gentle, non-toxic and effective. In this manner, each of the medicinal materials 10 is processed to give out its most valuable essentials that are helpful to improve immunity, to enhance health and to cure diseases.

The extracts obtained after the extraction 30 are then mixed in a predetermined ratio into a mixture, or mixed in different ratios into various mixtures. The mixtures receive concentration 40 and form concentrates. After filling 41 and sterilization 50, the products 60, or the pharmaceutical compositions, are ready to serve for meeting different needs.

As described previously, in one embodiment, the product 60 of the made of the medicinal materials 10 including Parispolyphylla Smith varchinensis(Franch.)Hara, Hedyotis diffusa (white flower snake-tongue grass), Mucuna macrocarpa Wallich, Pseudobulb of Appendiculate Cremastra, Solanum lyratumThunb (BittersweetHerb), Glehnia littoralis Fr.Schmidt ex Miq, Semiaquilegia adoxoides (DC.)makino., and Baphicacanthus cusia (Nees) Bremek. (Natural Indigo) as the main ingredients thereof. The product 60 has been proven to be curatively effective to all hematologic cancers, such as leukemia, primary brain tumors, osteocarcinoma, and prostate cancer.

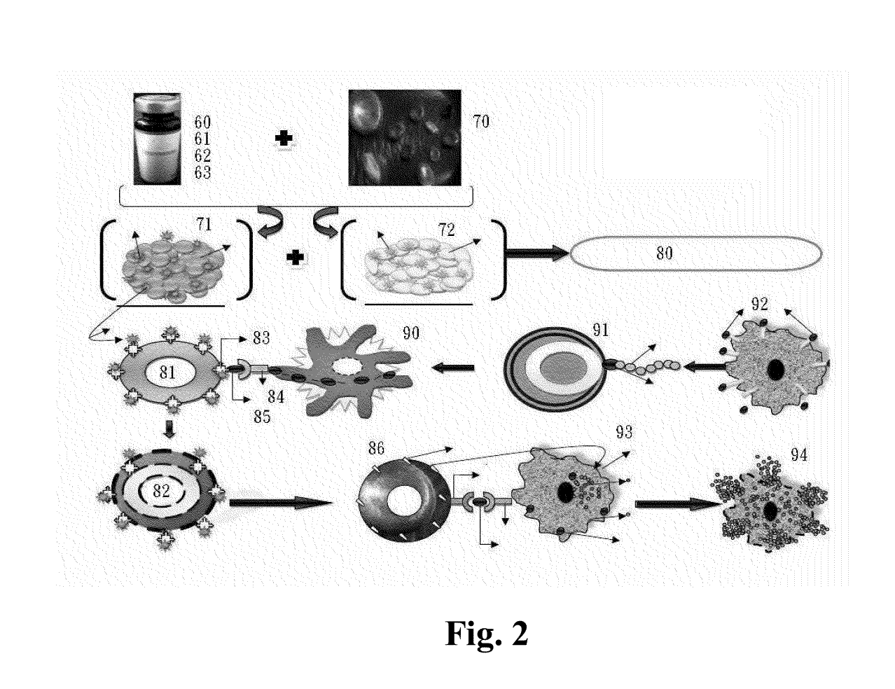

FIG. 2 illustrates the mechanism of the present invention. In use, the inventive product 60 has its active ingredients carried by rhodocytes 71 circulated in human blood 70, thereby helping leukocytes 72 to improve the performance of body apparatuses 80. Also, rhodocytes 71 can carry hydrophilic active molecules to make them attach to the surfaces of T cells 81. As a result, the active molecules in the pharmaceutical composition can activate the T cells 81, to generate activated T cells 82. Meanwhile, T cell's receptor 83 and the major histocompatibility complex 84 and the antigen 85 act on the antigen presenting cells 90, so as to make the antigen-presenting cell precursors 91 and tumor antigen-related cytokines 92 change. The activated T cells 82 can secrete cytokines and generate cytotoxic T cells 86 that attack tumor cells 93. Further, granzyme acts to break the tumor cells 93, forming disagglomerated tumor cells 94. Thereby, the inventive product 60 is useful to improve human immunity. When taken into body by the macrophages of the small intestines, it presents powerful catalyzing effects on the receptors of the membrane of most human cells, so as to make some actions taking place in cell nucleus stronger, thereby accomplishing immunotherapy. (See Appendix I, FR-AC00384 Rat Orally Administrated Acute Toxicity Test CHSG-608, and Appendix II FR-SC00031-Final Rat 13-Week Appendix Repeated Dose (Subchronic) Toxicity Test CHSG-608).

To sum up, the present invention is accomplished by selecting various natural medicinal materials and implementing immersion, extraction, concentration, and other processes, so as to prepare pharmaceutical products for different immunotherapy-related treatments, thereby preventing, mitigating and curing hematologic diseases. The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A method for preparation of an orally administrable pharmaceutical composition containing traditional Chinese medicine for hematologic diseases, the method comprising: selecting medicinal materials from traditional Chinese medicine; performing primary processing on the medicinal materials according to their respective properties; immersing the medicinal materials in a base liquor separately according to their respective properties so as to make the medicinal materials release their active substances in respective soaks thereof; promoting disagglomeration so as to make fermentable substances in the base liquor to balance the active substances of each of the medicinal materials; performing extraction on the soaks; mixing the soaks in a predetermined ratio into a mixture; performing concentration on the mixture so as to form a concentrate; and performing sterilization and bottle-filling on the concentrate, so as to obtain the orally administrable pharmaceutical composition that helps to enhance and activate immunocytes, thereby providing immunotherapy for the hematologic diseases.

2. The method of claim 1, wherein the product is prepared using the medicinal materials including Parispolyphylla Smith var. chinensis (Franch.) Hara, Hedyotis diffusa, Mucuna macrocarpa Wallich, Pseudobulb of Appendiculate Cremastra, Solanum lyratumThunb, Glehnia littoralis Fr.Schmidt ex Miq, Semiaquilegia adoxoides (DC.) makino, and Baphicacanthus cusia (Nees) Bremek, and has curative effects on hematologic cancers, including leukemia, primary brain tumors, osteocarcinoma and prostate cancer.
